(19) United States
(12) Patent Application Publication
(10) Pub. No.: US 2025/0011257 A1
(43) Pub. Date: Jan. 9, 2025

(54) PROCESS FOR PRODUCING OLEFINS AND BTX AROMATICS FROM OXYGENATES

(71) Applicant: L'Air Liquide, Societe Anonyme pour l'Etude et l’Exploitation des Procedes Georges Claude, Paris (FR)

(72) Inventors: Stephane HAAG, Frankfurt am Main (DE); Thomas RENNER, Frankfurt am Main (DE); Lin LIN, Frankfurt am Main (DE); Bryce WILLIAMS, Frankfurt am Main (DE)

(73) Assignee: L'Air Liquide, Societe Anonyme pour l'Etude et l’Exploitation des Procedes Georges Claude, Paris (FR)

(21) Appl. No.: 18/763,650

(22) Filed: Jul. 3, 2024

(30) Foreign Application Priority Data
Jul. 6, 2023 (EP) 23183865

(51) Int. Cl. C07C 1/20

(52) U.S. Cl. CPC C07C 1/20; C07C 2529/40

(57) ABSTRACT

The invention relates to a process and to a plant for preparation of olefins from oxygenates (OTO), where it is simultaneously also the intention to optimize the yield of the BTX aromatics benzene, toluene and the isomeric xylenes. According to the invention, for this purpose, the gasoline fraction obtained in the OTO process according to the prior art is added to an aromatization reactor. Multistage separation of the product stream from the aromatization reactor affords a product stream containing BTX aromatics and a gasoline fraction that has been depleted of olefins, cycloalkanes and cycloalkenes.

### CROSS-REFERENCE TO RELATED APPLICATIONS

This application claims the benefit of priority under 35 U.S.C. § 119(a) and (b) to EP patent application No. EP23183865, filed Jul. 6, 2023, the entire contents of which are incorporated herein by reference.

### BACKGROUND

### Field of the Invention

The invention relates to a process and to a plant for preparation of olefins from oxygenates (OTO), where it is simultaneously also the intention to optimize the yield of the BTX aromatics benzene, toluene and the isomeric xylenes.

### Related Art

Short-chain olefins, for example propylene (propene) and ethylene (ethene), are among the most important commodities in the chemical industry. The reason for this is that, proceeding from these unsaturated compounds with a short chain length, it is possible to form molecules having a long-chain carbon skeleton and additional functionalizations. These short-chain olefins find wide use particularly in the production of plastics by polymerization.

The main source of short-chain olefins in the past was steamcracking, i.e. thermal cracking in mineral oil processing. In the past few years, however, further processes for preparing short-chain olefins have been developed. One reason for this is rising demand that can no longer be covered by the available sources; secondly, the increasing scarcity of fossil raw materials is requiring the use of different starting materials.

What are called the MTP (methanol-to-propylene) or else MTO (methanol-to-olefin) processes for producing propylene and other short-chain olefins proceed from methanol as starting material. By way of generalization, reference is also made in this context to oxygenate-to-olefin (OTO) processes, since oxygen-containing organic components such as methanol or dimethyl ether (DME) are also referred to as oxygenates. In these heterogeneously catalysed processes, accordingly, there is at first partial formation of the dimethyl ether intermediate from methanol for example, and subsequently of a mixture of ethylene and propylene and hydrocarbons having higher molar mass, including olefins, from a mixture of methanol and dimethyl ether. Moreover, water is present in the product stream, which firstly originates from the process steam which is optionally supplied to the MTO reactor for modulation of reaction and secondly from the water of reaction produced in the MTP reactor, which is formed as a coproduct of the olefin formation reaction.

The subsequent purification is intended firstly to remove unwanted by-products and unconverted reactants, and to prepare the individual hydrocarbon fractions with maximum purity. Typically, for this purpose, a quench system is employed in the first step. Quenching here means abrupt or shock cooling which is usually brought about by direct heat exchange with a fluid quench medium. If a liquid such as water or methanol is used for the purpose, there is additionally a certain cleaning effect in relation to the remaining gas phase.

One example of the purification that follows an OTO reaction can be found in patent publication DE 10 2014 112 792 A1, which describes how, in a first step, a heterogeneously catalysed conversion of at least one oxygenate to a product stream comprising C2 olefins, C3 olefins, C4 olefins, C5/6 hydrocarbon compounds and C7+ hydrocarbon compounds and, in a second step, a removal of a propylene stream consisting to an extent of at least 95% by weight of C3 olefins is generated.

The further purification units that are described in DE 10 2014 112 792 A1 are in accordance with the concept customary in the art. The quenching may already bring about a coarse separation of the fractions according to their chain length of the resultant olefins due to partial condensation, thus allowing a liquid C4+ fraction to be discharged from the quench. The C4− fraction separated in gaseous form is subsequently fed into a compression stage. The C4− fraction from the compression is then sent to a separation apparatus in which C3− hydrocarbons are separated from the C4+ hydrocarbons. In subsequent purifying steps, the C3 fraction is separated from the C2− fraction in a further separating unit, which has to be effected under pressure owing to the low boiling points of the two fractions. Overall, the purification of the product stream is complicated since the aim is a high purity of the products. This is true of MTP plants having typical production capacities of about 470 kta of propylene, but becomes even more crucial if the plant capacity is lower (100 kta or 200 kta of propylene). This means that small plants are relatively uneconomic.

Overall, the products of the MTP process include a propylene product stream, an ethylene product stream and a gasoline fraction, the latter comprising higher paraffins, olefins, aromatics and cyclic hydrocarbons. Moreover, various hydrocarbons are obtained within the process, in particular streams containing C4 olefins, C5 olefins and C6+ olefins, which are recycled predominantly or completely to the MTP reactor, where they are cleaved to short-chain olefins, which improves the yield of the propylene and ethylene target products.

The main aim of the MTP process in its existing configuration is the maximization of the yield of propylene, which is a particularly sought-after product. However, there is also a need to increase the yield of other materials of value, for example of aromatic hydrocarbons, especially of BTX aromatics (benzene, toluene, xylenes), without significantly reducing the yield of the primary target products propylene and ethylene.

For this purpose, the configurations of the MTP process and of other OTO processes that are known from the prior art do not offer a satisfactory solution to date.

### SUMMARY OF THE INVENTION

It is therefore an object of the present invention to propose a process and a plant for preparation of short-chain olefins from oxygenates, especially ethylene and propylene, with additional production of BTX aromatics, which avoid the disadvantages of OTO processes that have been mentioned and are known to date from the prior art. This object is achieved in a first aspect of the invention by a process having the features of Claim 1 and in a further aspect of the invention by a plant having the features of Claim 10. Further aspects will be apparent from the dependent process claims.

The oxygenate conversion conditions required for the conversion of oxygenates to olefin products are known to the person skilled in the art from the prior art, for example the publications discussed in the introduction. These are those physicochemical conditions under which a measurable conversion, preferably one of industrial relevance, of oxygenates to olefins is achieved. Necessary adjustments of these conditions to the respective operational requirements will be made by those skilled in the art on the basis of routine experiments. Any specific reaction conditions disclosed may serve here as a guide, but they should not be regarded as limiting in relation to the scope of the invention.

Thermal separation methods for the purposes of the present invention include all separation methods based on the establishment of a thermodynamic phase equilibrium. Distillation or rectification are preferred. In principle, however, the use of other thermal separation methods is also conceivable, for example of extraction or extractive distillation.

If it is stated that a stream comprises hydrocarbons and comprises specifically olefins, this shall be considered to mean that the olefins are a specific subgroup of the hydrocarbons and that, accordingly, other non-olefinic hydrocarbons may also be present in the stream.

Oxygenates in principle mean all oxygen-containing hydrocarbon compounds that can be converted under oxygenate conversion conditions to olefins, especially to short-chain olefins such as propylene, and further hydrocarbon products. Examples are methanol or dimethyl ether (DME).

Short-chain olefins in the context of the present invention in particular mean olefins that are gaseous under ambient conditions, for example ethylene, propylene and the isomeric butenes 1-butene, cis-2-butene, trans-2-butene, isobutene.

Higher hydrocarbons or longer-chain hydrocarbons in the context of the present invention in particular mean hydrocarbons that are liquid in pure form under ambient conditions.

Hydrocarbon fractions are identified using the following nomenclature: “Cn fraction” refers to a hydrocarbon fraction containing predominantly hydrocarbons of carbon chain length n, i.e. having n carbon atoms. “Cn− fraction” refers to a hydrocarbon fraction containing predominantly hydrocarbons of carbon chain length n but also containing shorter carbon chain lengths. “Cn+ fraction” refers to a hydrocarbon fraction containing predominantly hydrocarbons of carbon chain length n but also containing longer carbon chain lengths. Owing to the physical separation methods used, for example distillation, separation in terms of carbon chain length should not be considered to mean that hydrocarbons having another chain length are rigorously excluded. For instance, a Cn− fraction, depending on the process conditions of the separation method, will still contain small amounts of hydrocarbons having a carbon number greater than n.

The solid, liquid and gaseous/vaporous states of matter mentioned should always be considered in relation to the local physical conditions that exist in the respective process step or in the respective plant component, unless stated otherwise. In the context of the present patent application, the gaseous and vapour states of matter should be considered to be synonymous.

In the context of the present invention, a division or separation/removal of a stream of matter means production of at least two substreams from the original stream of matter, where separation/removal is associated with an intentional alteration of the physical composition of the substreams obtained relative to the original stream of matter, for example by application of a thermal separation method or at least thermal separation step to the original stream of matter. By contrast, division of the original stream of matter is generally not associated with any change in the physical composition of the substreams obtained.

A gasoline fraction means a substance mixture which is in liquid form under ambient conditions, consists predominantly, preferably substantially completely, of higher hydrocarbons and may be suitable for use as a gasoline fuel.

The predominant part of a fraction, of a stream of matter, etc. means a proportion quantitatively greater than each of the other proportions on their own. Especially in the case of binary mixtures or in the case of separating of a fraction into two portions, this is understood to mean a proportion of more than 50% by weight, unless stated otherwise in the specific case.

The statement that a stream of matter consists predominantly of one component or group of components is understood to mean that the molar proportion (mole fraction) or proportion by mass (mass fraction) of this component or component group is quantitatively greater than each of the other proportions of other components or component groups in the stream of matter on their own. Especially in the case of binary mixtures, this is understood to mean a proportion of more than 50%. Unless stated otherwise in the specific case, the basis used here is the proportion by mass (mass fraction).

According to the invention, for this purpose, the gasoline fraction obtained in the MTP process according to the prior art (“MTP gasoline”) is added to an aromatization reactor. In one example, the aromatization reactor is constructed as a fixed bed reactor and is filled with a bed of a solid, particulate aromatization catalyst. In one example, the aromatization reactor is operated in straight pass without recycling of a product substream.

According to the invention, the product stream from the aromatization reactor is cooled and partly condensed, and then introduced into a phase separation apparatus. A gaseous stream of matter G2 is discharged from the phase separation apparatus and, in one example, is fed wholly or partly to the compressor. In addition, a liquid stream of matter A is obtained, from which a product stream containing BTX aromatics is ultimately obtained by workup in several steps by thermal separation methods.

First of all, the liquid stream of matter A is fed to a depentanizer column in which it is separated into a depentanizer top product stream comprising C5− hydrocarbons and a depentanizer bottom product stream comprising C6+ hydrocarbons.

The depentanizer bottom product stream is fed to an aromatics separation apparatus that works by at least one thermal separation method, for example distillation or extractive distillation or combinations thereof, and, in one example, comprises several separation columns connected in series. Products obtained from the aromatics separation apparatus are a first product stream containing nonaromatic hydrocarbon and a second product stream containing BTX aromatics. This is discharged from the process as BTX aromatics product stream. Depending on the specific type of separation methods used, the first and/or second product stream may each be obtained as top product stream, bottom product stream or sidestream from the corresponding separation columns.

It is particularly advantageous here that the invention affords BTX aromatics as additional materials of value in good yields without any reduction in the yield of the primary target products ethylene and propylene, since the BTX aromatics are obtained by further processing of the gasoline fraction from the MTP process according to the prior art. There is a trend toward slightly increasing yield of the primary target products ethylene and propylene, since treatment of the gasoline fraction in the aromatization reactor produces components as secondary products that can be fed to the pool of primary target products.

In addition, the invention improves the usability of the gasoline fraction: Because of its high content of higher or longer-chain olefins and of cycloalkanes and cycloalkenes, the gasoline fraction obtained in the MTP process according to the prior art is not directly usable as gasoline fuel. But the inventive treatment of the gasoline fraction in the aromatization reactor distinctly reduces the concentration of these problematic components, since they are converted to aromatics for the most part. After the aromatics have been separated off, what remains is a converted gasoline fraction that can be added without any problem to the hydrocarbon pool for the production of gasoline fuels.

### Further Aspects of the Invention

A second aspect of the invention is characterized in that, in the process, at least a portion of the gaseous stream of matter G2 is introduced into the compressor. Since reactive components such as olefins are still present in the gaseous stream of matter G2, it is possible in this way to further increase the yield of the short-chain olefins ethylene and propylene.

A third aspect of the invention is characterized in that, in the process, at least a portion of the depentanizer top product stream is returned to and introduced into the compressor. Since reactive components such as olefins are still present in the depentanizer top product stream, it is possible in this way to further increase the yield of the short-chain olefins ethylene and propylene.

A fourth aspect of the invention is characterized in that, in the process, the portion of the depentanizer top product stream returned to the compressor is mixed with the reactor product stream before being introduced into the compressor. In this way, a more uniform feedstock stream is obtained for the compressor, and fluctuations in concentration are avoided. This is important especially in the case of non-steady states of the process, for example startup operations, shutdown operations or changes in load.

A fifth aspect of the invention is characterized in that, in the process, the aromatization conditions comprise a reactor temperature between 380 and 510° C., a reactor pressure between 1 and 6 bara, and the presence of a solid aromatization catalyst. Studies have shown that these aromatization conditions are particularly suitable for achieving a high yield of BTX aromatics.

A sixth aspect of the invention is characterized in that, in the process, the aromatization catalyst is present in the aromatization reactor in the form of a fixed bed of a granular catalyst, preferably a zeolite catalyst, most preferably a zeolite catalyst of the ZSM-5 structure type. Aromatization catalysts of this type are tried and tested, and commercially available.

A seventh aspect of the invention is characterized in that, in the process, the aromatization catalyst used is the OTO catalyst. In this way, technical and logistical advantages are obtained, since, for example, the storage of fresh or spent catalyst is simplified.

An eighth aspect of the invention is characterized in that, in the process, the aromatization catalyst used is a batch of the OTO catalyst that has already been used in the OTO reactor beforehand. Studies show that the catalyst already used in the OTO reactor still has sufficient activity as aromatization catalyst. Therefore, resources are conserved and economic benefits are obtained.

A ninth aspect of the invention is characterized in that, in the process, the aromatization catalyst used is a batch of the OTO catalyst that has already been used in the OTO reactor beforehand during several cycles with intermediate regeneration. The conservation of resources and economic benefits are enhanced here once again.

An eleventh aspect of the invention is characterized in that the plant further comprises means which permit introduction of at least a portion of the gaseous stream of matter G2 into the compressor. The advantages associated with this aspect correspond to those elucidated in connection with the second aspect of the invention.

A twelfth aspect of the invention is characterized in that the plant further comprises means that permit returning of at least a portion of the depentanizer top product stream to the compressor and introduction thereof into said compressor. The advantages associated with this aspect correspond to those elucidated in connection with the third aspect of the invention.

A thirteenth aspect of the invention is characterized in that, in the plant, the aromatization catalyst is present in the aromatization reactor in the form of a fixed bed of a granular catalyst, preferably a zeolite catalyst, most preferably a zeolite catalyst of the ZSM-5 structure type. The advantages associated with this aspect correspond to those elucidated in connection with the sixth aspect of the invention.

A fourteenth aspect of the invention is characterized in that, in the plant, the aromatization catalyst used is the OTO catalyst. The advantages associated with this aspect correspond to those elucidated in connection with the seventh aspect of the invention.

A fifteenth aspect of the invention is characterized in that, in the plant, the aromatization catalyst used is a batch of the OTO catalyst that has already been used in the OTO reactor beforehand. The advantages associated with this aspect correspond to those elucidated in connection with the eighth aspect of the invention.

A sixteenth aspect of the invention is characterized in that, in the plant, the aromatization catalyst used is a batch of the OTO catalyst that has already been used in the OTO reactor beforehand during several cycles with intermediate regeneration. The advantages associated with this aspect correspond to those elucidated in connection with the ninth aspect of the invention.

### BRIEF DESCRIPTION OF THE FIGURES

Further features, advantages and possible applications of the invention are apparent from the following description of working and numerical examples and from the drawings. All the features described and/or depicted, on their own or in any combination, form the subject-matter of the invention, irrespective of their combination in the claims or their dependency references.

The figures show:

FIG. 1 a schematic diagram of a process/plant according to the prior art,

FIG. 2 a schematic diagram of a process/plant according to the invention.

What is meant by “not shown” hereinafter is that an element in the figure under discussion is not graphically represented but nevertheless present in accordance with the description.

### DETAILED DESCRIPTION OF THE INVENTION

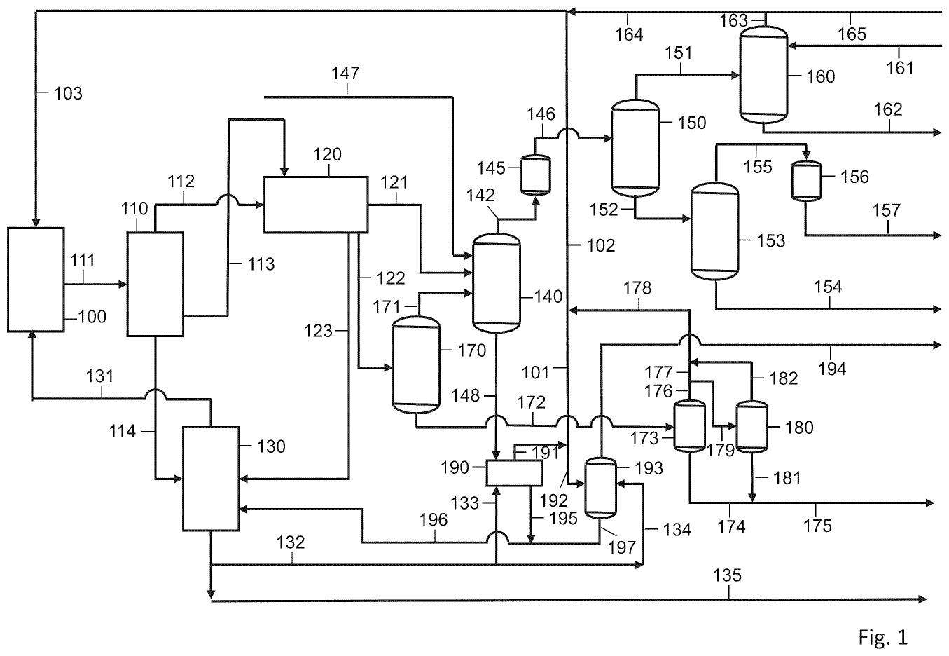

FIG. 1 shows a schematic diagram of an MTP process and of an MTP plant for preparation of ethylene and propylene according to the prior art.

The OTO reactor 100 in which the formation of olefins from oxygenates, for example methanol and/or dimethyl ether (DME), proceeds is charged via a fresh feed conduit (not shown) with oxygenates and water vapour as diluent; in addition, recycle streams are also returned to the reactor via conduits 103 and 131. Via conduit 111, the product stream from the reactor 100 is introduced into the quench system 110. A phase containing essentially water is removed from the quench system 110 via conduit 114 and introduced into a methanol recovery unit 130. The gaseous phase removed in the quench system 110 enters a compressor 120 via conduit 112. Via conduit 113, a liquid phase consisting essentially of hydrocarbons is additionally fed from the quench system 110 into the compressor 120.

From the compressor 120, a liquid stream is conducted via conduit 121 into a separation apparatus 140. In this separation apparatus 140, the C3− fraction is separated from the C4 fraction, which is why it is also referred to as depropanizer. The separation apparatus 140 is preferably configured as a separation column. The rectification in the separation apparatus 140 is preferably effected as an extractive distillation, and it is therefore possible to supply an extractant, for example methanol, via conduit 147 if desired.

The top product drawn off overhead, containing the C3− fraction, is fed via conduit 142 to a dryer 145, from which it goes on to enter a further separation apparatus 150 via conduit 146. A direct connection of separation apparatus 140 to separation apparatus 150 is also possible. In this separation apparatus 150, the C3 fraction is separated from the C2− fraction, which is why the separation apparatus 150 is also referred to as deethanizer.

Via conduit 151, the top product from the separation apparatus 150 enters a separation apparatus 160, preferably in the form of a scrubber. When it is in the form of a scrubber, a scrubbing agent, for example scrubbing water, is introduced via conduit 161 and drawn off again via conduit 162. The C2− stream present is drawn off overhead by conduit 163 and can be sent via conduit 164 to a recycle stream in conduit 103 that leads back into the reactor 100 and/or discharged from the system via conduit 165 and sent to a further separation apparatus (not shown) for purification of the products present in order to obtain, for example, ethylene present as pure product.

The bottom product from the separation apparatus 150 is fed via conduit 152 to a separation apparatus 153 that separates propane from propylene. In this so-called C3 splitter, propane is drawn off from the system via conduit 154, and this can find use as fuel gas, for example. The propylene target product is drawn off via conduit 155, and this can optionally, depending on quality demands, be sent to a further purifying apparatus 156, from which it is then discharged via conduit 157. The purification apparatus 156 may be filled, for example, with a sorbent selected for oxygenates, such that oxygenates can be removed down to small traces from the propylene product stream according to the specification.

A liquid phase is drawn off from the compressor via conduit 122, and this is sent to a separation apparatus 170 in which the C4− fraction and the oxygenates are separated from the C4+ fraction. This separation apparatus 170 is also referred to as debutanizer. Via conduit 171, the C4− fraction and the oxygenate-containing top product from the separation apparatus 170 enter the separation apparatus 140.

Via conduit 172, the bottom product from the separation apparatus 170 is introduced into a further separation apparatus 173 in which the C7+ fraction is separated from the C6 fraction, which is why the separation apparatus 173 is also referred to as dehexanizer. The bottom product is drawn off via conduit 174, 175. Via conduit 176, 177 and 178, a recycle stream can be sent to a conduit 101, which leads via conduits 102 and 103 back to the reactor 100.

Moreover, a conduit 179 may be branched off from conduit 176, and this leads into a separation apparatus 180 (gasoline stabilizer column). The gasoline obtained here is discharged as product of value via conduit 181 and conduit 175. The C4 hydrocarbons removed overhead are fed via conduit 182 to conduits 177 and 178, such that they ultimately open into the recycle stream in conduits 102, 103.

The bottom product from the separation apparatus 140 is drawn off via conduit 148 and thus enters a mixer-settler combination 190. Hydrocarbons are drawn off therefrom via conduit 191 and can be returned wholly or partly via conduits 101, 102, 103 to the reactor 100. Alternatively or additionally, it is possible to send the hydrocarbons via conduit 192 to a separation apparatus 193 which is preferably configured as an extraction. The top product from the separation apparatus 193 is discharged as fuel gas via conduit 194.

From the methanol-dimethyl ether recovery 130, an oxygenate recycle stream is firstly returned via conduit 131 to the reactor 100. Via conduits 132 and 133, it is optionally possible to feed methanol- and/or dimethyl ether-laden water into the mixer-settler unit 190, from which it can then also be fed via conduit 195 back to conduits 197 and 196.

Via conduits 132, 134, the water can be conducted as extractant into the separation apparatus 193 if it is configured as an extraction, from which it can then also be fed via conduit 197 back to conduit 196. The water can ultimately be discharged from the system via conduit 135.

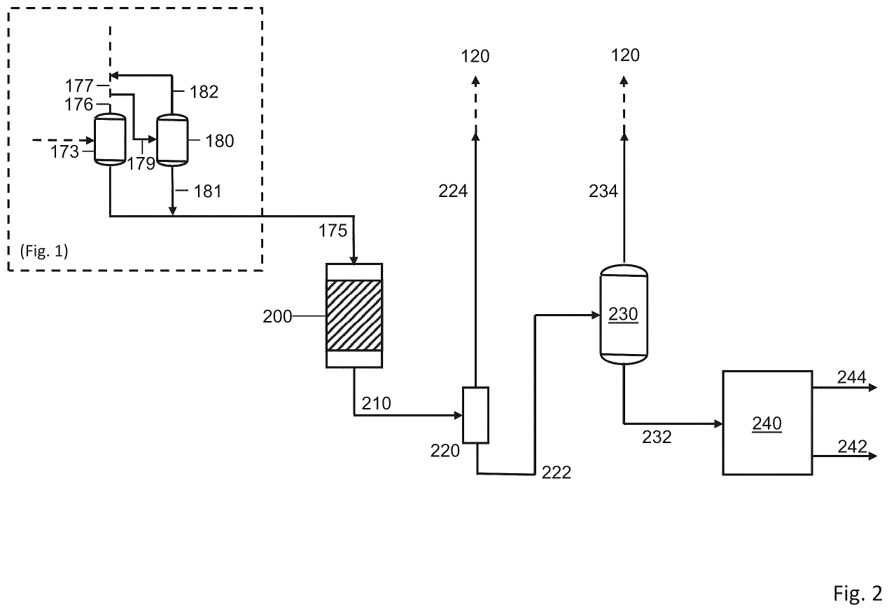

According to FIG. 2, in accordance with the invention, the dehexanizer bottom product stream (“MTP gasoline”) discharged via conduit 175, after preheating and/or evaporation (not shown), is fed to an aromatization reactor 200 and converted therein under aromatization conditions to an aromatization reactor product stream enriched with aromatic hydrocarbons, especially with BTX aromatics. In one example, the aromatization conditions comprise a reactor temperature between 380 and 510° C., a reactor pressure between 1 and 6 bara, and the presence of a solid aromatization catalyst. In one example, the aromatization catalyst is present in the aromatization reactor in the form of a fixed bed of a granular catalyst, preferably a zeolite catalyst, most preferably a zeolite catalyst of the ZSM-5 structure type. In one example, the aromatization catalyst used is the OTO catalyst. In one example, the aromatization catalyst used is a batch of the OTO catalyst that has already been used in the OTO reactor beforehand. In one example, the aromatization catalyst used is a batch of the OTO catalyst that has already been used in the OTO reactor beforehand during several cycles with intermediate regeneration.

The gaseous aromatization reactor product stream discharged from the aromatization reactor is cooled down and partly condensed (neither of which is shown) and then introduced into a phase separation apparatus 220 via a conduit 210. A gaseous stream of matter G2 is discharged therefrom, which is discharged via a conduit 224. In one example, the gaseous stream of matter G2 is returned at least partly to the compressor 120 via conduit 224. In addition, a liquid stream of matter A is discharged from the phase separation apparatus 220 via a conduit 222 and introduced into a depentanizer column 230.

A depentanizer top product stream comprising C5− hydrocarbons is discharged from the depentanizer column 230 via a conduit 234. In one example, the depentanizer top product stream is returned at least partly to the compressor 120 via conduit 234. In addition, via a conduit 232, a depentanizer bottom product stream comprising C6+ hydrocarbons is discharged from the depentanizer column 230 and introduced at least partly into an aromatics separation apparatus 240 that works by at least one thermal separation method.

In the aromatics separation apparatus 240, the depentanizer bottom product stream is separated into a first product stream containing nonaromatic hydrocarbon and discharged from the process or plant via a conduit 244, and a second product stream that contains BTX aromatics and is discharged from the process or plant as BTX aromatics product stream via a conduit 242. In one example, the aromatics separation apparatus 240 comprises at least two thermal separation methods. In one example, the at least two thermal separation methods comprise distillation, rectification, extractive distillation, extraction. The selection and use of these thermal separation methods and the selection of suitable operating parameters is known per se to the person skilled in the art and/or can be ascertained by them by routine experiments without difficulties.

### Numerical Examples

An MTP gasoline stream of 540 tonnes per day (tpd) was worked up according to the invention. The results obtained were compiled in the tables that follow.

TABLE 1
Composition of the MTP gasoline (comparative)

| Comparison | | | | |
|---|---|---|---|---|
| (FIG. 1) | Olefins | Paraffins | Naphthenes | Aromatics |
| wt % | 22.3 | 32.5 | 19.0 | 26.2 |
| tpd | 120.42 | 175.5 | 102.6 | 141.48 |

TABLE 2
Composition of the MTP gasoline after
workup according to invention

| Invention | | | | |
|---|---|---|---|---|
| (FIG. 2) | Olefins | Paraffins | Naphthenes | Aromatics |
| wt % | 8.6 | 39.1 | 8.6 | 43.7 |
| tpd | 46.13 | 211.21 | 46.55 | 236.12 |

TABLE 3
Composition of the MTP gasoline (comparative): Olefins

| Comparison | Ethylene | Propylene | Butylene |
|---|---|---|---|
| tpd (in gasoline) | 0 | 0 | 2.7 |

TABLE 4
Composition of the MTP gasoline after
workup according to invention: Olefins

| Invention | Ethylene | Propylene | Butylene |
|---|---|---|---|
| tpd (in gasoline) | 5.8 | 11.4 | 4.8 |

TABLE 5
Composition of the MTP gasoline (comparative): BTX aromatics

| Comparison | Benzene | Toluene | Xylenes |
|---|---|---|---|
| tpd (in gasoline) | 1.1 | 19.4 | 80.7 |

TABLE 6
Composition of the MTP gasoline after workup
according to invention: BTX aromatics

| Invention | Benzene | Toluene | Xylenes |
|---|---|---|---|
| tpd (in gasoline) | 11.6 | 57.4 | 108.3 |

It is clearly apparent from the data compiled in the tables that the inventive workup of the MTP gasoline results in:
a distinct increase in the content and the amount of product of aromatics,
a decrease in the content of olefins, especially longer-chain olefins, and of naphthenes (cycloalkanes) in the MTP gasoline,
an increase in the content and the amount of product of short-chain olefins, especially of ethylene and propylene,
a distinct increase in the content and the amount of product of BTX aromatics.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed. Furthermore, if there is language referring to order, such as first and second, it should be understood in an exemplary sense and not in a limiting sense. For example, it can be recognized by those skilled in the art that certain steps can be combined into a single step.

The singular forms “a”, “an” and “the” include plural referents, unless the context clearly dictates otherwise.

“Comprising” in a claim is an open transitional term which means the subsequently identified claim elements are a nonexclusive listing i.e. anything else may be additionally included and remain within the scope of “comprising.” “Comprising” is defined herein as necessarily encompassing the more limited transitional terms “consisting essentially of” and “consisting of”; “comprising” may therefore be replaced by “consisting essentially of” or “consisting of” and remain within the expressly defined scope of “comprising”.

“Providing” in a claim is defined to mean furnishing, supplying, making available, or preparing something. The step may be performed by any actor in the absence of express language in the claim to the contrary.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within said range.

All references identified herein are each hereby incorporated by reference into this application in their entireties, as well as for the specific information for which each is cited.

### LIST OF REFERENCE NUMERALS

100 OTO reactor
101-103 conduit
110 quench system
111-114 conduit
120 compressor
121-123 conduit
130 methanol-DME recovery
131-135 conduit
140 separation apparatus
142 conduit
150 separation apparatus
151, 152 conduit
153 separation apparatus
154, 155 conduit
156 purification stage
157 conduit
160 separation apparatus
161-165 conduit
170 separation apparatus
171, 172 conduit
173 separation apparatus
174-179 conduit
180 separation apparatus (gasoline stabilizer column)
181, 182 conduit
190 mixer-settler combination
191,192 conduit
193 separation apparatus
194-197 conduit
200 aromatization reactor
210 conduit
220 phase separation apparatus
222 conduit
224 conduit
230 depentanizer column
232 conduit
234 conduit
240 aromatics separation apparatus
242 conduit
244 conduit

1. A process for preparing ethylene, propylene and BTX aromatics from oxygenates, comprising the following steps:
(a) providing a feed stream containing oxygenates and converting the oxygenates in the feed stream under heterogeneous catalysis in an oxygenate-to-olefin reactor (OTO reactor) over an oxygenate-to-olefin catalyst (OTO catalyst) under oxygenate conversion conditions to a reactor product stream containing water, hydrocarbons and at least one oxygenate;
(b) quenching the reactor product stream in at least one quench stage and compressing the quenched reactor product stream in a compressor downstream of the quench stage, wherein the quenching and/or compressing of the reactor product stream affords the following streams of matter:
(b1) a gaseous stream of matter G1 comprising hydrocarbons and comprising specifically olefins,
(b2) a liquid stream of matter W comprising water and oxygenates,
(b3) a liquid stream of matter O comprising hydrocarbons and comprising specifically olefins;
(c) multistage separation of stream of matter G1 by thermal separation methods to obtain a group of product streams, where the group of product streams comprises:
(c1) an ethylene-containing ethylene product stream which is discharged at least partly from the process,
(c2) a propylene-containing propylene product stream which is discharged from the process;
(d) multistage separation of stream of matter O by thermal separation methods, wherein
(d1) stream of matter O is fed to a debutanizer column and separated into a debutanizer top product stream and a debutanizer bottom product stream therein, where the debutanizer top product stream comprises C4− hydrocarbons and oxygenates and where the debutanizer bottom product stream comprises C4+ hydrocarbons,
(d2) the debutanizer top product stream is sent to step (c),
(d3) the debutanizer bottom product stream is introduced into a dehexanizer column;
(e) separating the debutanizer bottom product stream in the dehexanizer column into a dehexanizer top product stream comprising C6− hydrocarbons and a dehexanizer bottom product stream comprising C7+ hydrocarbons;
(f) recycling at least a portion, preferably the entirety, of the dehexanizer top product stream to the OTO reactor;
(g) evaporating at least a portion of the dehexanizer bottom product stream and introducing the evaporated dehexanizer bottom product stream into an aromatization reactor of fixed bed configuration, converting the evaporated dehexanizer bottom product stream in the aromatization reactor under aromatization conditions, discharging a gaseous aromatization reactor product stream from the aromatization reactor;
(h) cooling and partly condensing the gaseous aromatization reactor product stream and introducing the cooled aromatization reactor product stream into a phase separation apparatus, discharging a gaseous stream of matter G2 and a liquid stream of matter A from the phase separation apparatus;
(i) feeding at least a portion of the liquid stream of matter A to a depentanizer column that works by a thermal separation method, separating the liquid stream of matter A in the depentanizer column into a depentanizer top product stream comprising C5− hydrocarbons and a depentanizer bottom product stream comprising C6+ hydrocarbons;
(j) feeding at least a portion of the depentanizer bottom product stream to an aromatics separation apparatus that works by at least one thermal separation method, separating the depentanizer bottom product stream in the aromatics separation apparatus into a first product stream containing nonaromatic hydrocarbon and a second product stream containing BTX aromatics that is discharged from the process as BTX aromatics product stream.

2. The process of claim 1, w at least a portion of the gaseous stream of matter G2 is introduced into the compressor.

3. The process of claim 1, wherein at least a portion of the depentanizer top product stream is returned to and introduced into the compressor.

4. The process of claim 1, wherein the portion of the depentanizer top product stream returned to the compressor is mixed with the reactor product stream before being introduced into the compressor.

5. The process of claim 1, wherein the aromatization conditions comprise a reactor temperature between 380 and 510° C., a reactor pressure between 1 and 6 bara, and the presence of a solid aromatization catalyst.

6. The process of claim 5, wherein the aromatization catalyst is present in the aromatization reactor in the form of a fixed bed of a granular catalyst, preferably a zeolite catalyst, most preferably a zeolite catalyst of the ZSM-5 structure type.

7. The process of claim 6, wherein the aromatization catalyst used is the OTO catalyst.

8. The process of claim 7, wherein the aromatization catalyst used is a batch of the OTO catalyst that has already been used in the OTO reactor beforehand.

9. The process of claim 8, wherein the aromatization catalyst used is a batch of the OTO catalyst that has already been used in the OTO reactor beforehand during several cycles with intermediate regeneration.

10. A plant for preparation of ethylene, propylene and BTX aromatics from oxygenates, comprising the following assemblies and constituents in fluid connection to one another:
(a) an oxygenate-to-olefin reactor (OTO reactor) having an oxygenate-to-olefin catalyst (OTO catalyst), means of providing a feed stream containing oxygenates and means of introducing the latter into the OTO reactor, means of discharging a reactor product stream containing water, hydrocarbons and at least one oxygenate from the OTO reactor;
(b) a quench stage and a compressor downstream of the quench stage, configured for quenching of the reactor product stream in the quench stage and for compressing of the quenched reactor product stream in the compressor, means of discharging the following streams of matter from the quench stage and/or from the compressor:
(b1) a gaseous stream of matter G1 comprising hydrocarbons and comprising specifically olefins,
(b2) a liquid stream of matter W comprising water and oxygenates,
(b3) a liquid stream of matter O comprising hydrocarbons and comprising specifically olefins;
(c) a first multistage separation apparatus for separation of stream of matter G1 by thermal separation methods, means of discharging a group of product streams from the multistage separation apparatus, where the group of product streams comprises:
(c1) an ethylene-containing ethylene product stream which is at least partly discharged from the process, with recycling of a further fraction of the ethylene product stream to the OTO reactor,
(c2) a propylene-containing propylene product stream which is discharged from the process;
(d) a second multistage separation apparatus for separation of stream of matter O by thermal separation methods, further comprising means that permit the following:
(d1) stream of matter O is fed to a debutanizer column and separated into a debutanizer top product stream and a debutanizer bottom product stream therein, where the debutanizer top product stream comprises C4− hydrocarbons and oxygenates and where the debutanizer bottom product stream comprises C4+ hydrocarbons,
(d2) the debutanizer top product stream is sent to step (c),
(d3) the debutanizer bottom product stream is introduced into a dehexanizer column;
(e) means of separating the debutanizer bottom product stream in the dehexanizer column into a dehexanizer top product stream comprising C6− hydrocarbons and a dehexanizer bottom product stream comprising C7+ hydrocarbons;
(f) means of recycling at least a portion, preferably the entirety, of the dehexanizer top product stream to the OTO reactor;
(g) means of evaporating at least a portion of the dehexanizer bottom product stream and means of introducing the evaporated dehexanizer bottom product stream into an aromatization reactor of fixed bed configuration, means of discharging a gaseous aromatization reactor product stream from the aromatization reactor;
(h) a phase separation apparatus, means of cooling the gaseous aromatization reactor product stream and means of introducing the cooled aromatization reactor product stream into the phase separation apparatus, means of discharging a gaseous stream of matter G2 and a liquid stream of matter A from the phase separation apparatus;
(i) a depentanizer column that works by a thermal separation method, means of feeding at least a portion of the liquid stream of matter A to the depentanizer column, means of discharging a depentanizer top product stream comprising C5− hydrocarbons from the depentanizer column, and means of discharging a depentanizer bottom product stream comprising C6+ hydrocarbons from the depentanizer column;
(j) an aromatics separation apparatus that works by at least one thermal separation method, means of feeding at least a portion of the depentanizer bottom product stream to the aromatics separation apparatus, means of discharging a first product stream containing nonaromatic hydrocarbons from the aromatics separation apparatus, and means of discharging a second product stream containing BTX aromatics from the aromatics separation apparatus, and means of discharging the second product stream from the aromatics separation apparatus from the plant as BTX aromatics product stream.

11. The plant of claim 10, wherein it further comprises means which permit introduction of at least a portion of the gaseous stream of matter G2 into the compressor.

12. The plant of claim 10, wherein it further comprises means that permit returning of at least a portion of the depentanizer top product stream to the compressor and introduction thereof into said compressor.

13. The plant of claim 10, wherein the aromatization catalyst is present in the aromatization reactor in the form of a fixed bed of a granular catalyst, preferably a zeolite catalyst, most preferably a zeolite catalyst of the ZSM-5 structure type.

14. The plant of claim 10, wherein the aromatization catalyst used is the OTO catalyst.

15. The plant of claim 10, wherein the aromatization catalyst used is a batch of the OTO catalyst that has already been used in the OTO reactor beforehand.

16. The plant of claim 10, wherein the aromatization catalyst used is a batch of the OTO catalyst that has already been used in the OTO reactor beforehand during several cycles with intermediate regeneration.
